(19) United States
(12) Patent Application Publication
(10) Pub. No.: US 2010/0189647 A1
(43) Pub. Date: Jul. 29, 2010

(54) Experimental Animal As Pathological Model, Method of Producing the Experimental Animal, and Method of Using the Experimental Animal

(76) Inventors: Fusako Takayama, Okayama (JP); Toru Egashira, Okayama (JP)

Correspondence Address: LONDA, BRUCE S.;NORRIS MCLAUGHLIN & MARCUS, PA, 875 THIRD AVE, 8TH FLOOR, NEW YORK, NY, 10022, US

(21) Appl. No.: 12/377,045

(22) PCT Filed: Feb. 13, 2007
(86) PCT No.: PCT/JP2007/052477

§ 371 Date: Mar. 17, 2010

(30) Foreign Application Priority Data
Aug. 10, 2006 (JP) 2006-218825

(51) Int. Cl. A01K 67/027; A01K 67/00

(52) U.S. Cl. 424/9.1; 800/9

(57) ABSTRACT

Problem to be Solved: There are provided a novel pathological model with an experimental animal which reproduces human nonalcoholic chronic hepatitis and/or liver fibrosis and/or cirrhosis progressed from fatty liver, a method for producing the same, and a method for utilizing the novel pathological model with an experimental animal.

Solution: An in-vivo hypoxic state is formed in a fatty liver having model experimental animal, and a pathological model with an experimental animal, which keeps biochemical characteristics and/or histopathological characteristics of nonalcoholic steatohepatitis, and/or cirrhosis is finally produced.

### TECHNICAL FIELD

The present invention relates to a pathological model with an experimental animal having biochemical characteristics and/or histopathological characteristics of nonalcoholic steatohepatitis (hereinafter, referred to as “NASH”), a method for producing the same, and a method of utilizing the same.

### BACKGROUND ART

When inflammation occurs in addition to fatty liver and persists, the condition may progress to cirrhosis. NASH is a progressive liver disorder that occurs in patients without significant alcohol drinking history, and, histologically, it resembles alcoholic steatohepatitis. It is rapidly attracting attention as a new lifestyle-related disease along with hypertension, diabetes, and hyperlipemia.

NASH greatly differs from simple fatty liver in that steatohepatitis and liver fibrosis are present. Histopathological assessment is generally required to diagnose NASH (Non-Patent Document 2). This condition is characterized by macrovesicular steatosis, degeneration and spotty necrosis of hepatocytes, and periportal leukocytes infiltration, which result in fibrosis in the lobule. Due to excessive caloric intake and nutritive deflection, fatty liver similar to alcoholic hepatopathy progresses to steatohepatitis even without a drinking history, and liver fibrosis is aggravated due to this progression, ultimately leading to cirrhosis. The basic pathological conditions of cirrhosis are progressive irreversible loss of hepatic function (ascites, hemorrhagic tendency, and hepatic encephalopathy are induced by impairment in production of albumin and blood coagulation factors [including prothrombin]) and portal hypertension associated with a decreased portal vein blood flow (resulting in induction of esophageal varices, gastrointestinal tract hemorrhage, splenomegaly, hepatic encephalopathy, and the like). Furthermore, it is viewed as a problem that part of lesions become cancerous.

It is thought that, when a certain stimulus is applied to fatty liver, it progresses to steatohepatitis and liver fibrosis, and further to cirrhosis due to progression of severity. The onset mechanism of NASH is considered to involve fatty liver as a previous stage thereof. Fatty liver occurs first, then a certain stress is applied thereto, and it progresses to steatohepatitis, then further to a more advanced hepatopathy (cirrhosis) (Non-Patent Document 3). That is, it is inferred that a process in which fatty liver develops due to fat accumulation in the liver is a first stage of NASH, and fatty liver progresses to NASH, as a second stage, due to interactions with various mechanisms such as (a) oxidative stress (Non-Patent Documents 4 and 5), (b) induction of inflammatory cytokines by endotoxins, (c) expression and induction of CYP2E1 (Non-Patent Documents 6 and 7), and (d) abnormal function of mitochondria. Therefore, these stress loads on fatty liver are also required in the development of a NASH model with experimental animals. To produce a pathological model with an experimental animal based on the pathological condition in humans, stresses in human lifestyle habits that cause progression to the NASH condition or stress loads similar thereto are desired.

Along with increases in the obesity population or patients with lifestyle-related diseases due to the Westernized diet, specifically, a high fat content diet, and lack of exercises, it is anticipated that patients with fatty liver or NASH will also increase in number. Therefore, elucidation of the NASH condition, a progressive disease from fatty liver to hepatitis, liver fibrosis, and cirrhosis as well as development of new therapeutic agents for NASH, agents for preventing progression of severity, and/or functional foods for reducing a risk of developing NASH and functional foods for reducing a risk of progression of severity and/or establishment of methods for treatment of NASH or preventing progression of severity are awaited.

For elucidation of the NASH condition, a progressive disease as well as development of new agents for treatment of NASH, agents for preventing progression of severity, and/or functional foods for reducing a risk of developing NASH and functional foods for reducing a risk of progression of severity and/or establishment of methods for treating NASH or preventing progression of severity, it is very important to check how sites of hepatitis, liver fibrosis, and cirrhosis are affected. To this end, effects of materials that regulate physiological functions on pathological conditions classified into lifestyle-related diseases closely related to the progression of pathological conditions need to be observed over a long period using the pathological model with an experimental animal based on the human NASH condition.

However, all of the pathological models developed so far for elucidation of the mechanism of the NASH condition and drug treatment thereof based on nutritional findings have been only fatty liver models. Specifically, histopathological characteristics of the liver of animals with pathological conditions recently developed by giving a methionine or choline deficient feed or a feed with high fat and high carbohydrate components as NASH model animals (Non-Patent Documents 8 and 9) were only at a stage of fatty liver or moderate hepatitis, and no marked fibrosis was observed and, if any, it was very minor. These conditions were not similar to the NASH condition, which is very likely to progress from steatohepatitis to fibrosis. Furthermore, long-term breeding is required since a pathological model animal developed by giving a feed with high fat and high carbohydrate components (Non-Patent Document 10) develops fatty liver in about eight weeks and steatohepatitis in about 16 weeks.

Furthermore, models of pathological conditions such as hepatitis, liver fibrosis, and cirrhosis are conventionally produced by using agents for inducing chronic hepatitis or cirrhosis such as carbon tetrachloride, thioacetamide, and dimethylnitrosamine. These models have a common mechanism of developing hepatopathy. Specifically, these agents are all lipid-soluble substances, and are metabolized in the liver and converted to highly reactive and toxic metabolites. For example, carbon tetrachloride causes necrosis of the centrilobular region of the liver by a mechanism of metabolism in which carbon tetrachloride is converted to carbon trichloride radicals by the metabolism in the liver microsomal cytochrome P-450 enzymes, followed by production of trichloromethylperoxy radicals. Therefore, pathological conditions caused by steatohepatitis resulting from fatty liver essentially differ in the mechanism and the appearance from that of hepatitis induced by directly hepatotoxic agents mentioned above. (Non-Patent Document 11).

For production of a model mammal that keeps histopathological characteristics of chronic hepatitis and/or cirrhosis, a method of administering thioacetamide (Patent Document 1) is known, but is not realistic in view of cost and labor. The number of the model animals, which can be produced, is limited, and technical skills are required to increase reproducibility.

In normal life, humans are very unlikely to be exposed to agents inducing chronic hepatitis or cirrhosis, and pathological models produced thereby cannot be used as pathological models of steatohepatitis, liver fibrosis, cirrhosis, and liver cancer reflecting the NASH condition. Furthermore, for treatment of liver diseases, for example, methods using antioxidants (Patent Documents 2 and 3), methods using oxygen (Patent Document 4), and methods using L-alanine (Patent Document 5) are known. However, the pathological models do not have adequate similarity with humans, and these techniques are inadequate in view of the necessity of development of therapies and therapeutic agents based on analyses of the mechanism of advancing general lifestyle-related diseases including screening for and development of more improved prophylactic and therapeutic agents against progression of severity and the like.

Meanwhile, the risk of developing NASH in humans is increased in sleep apnea syndrome in patients with fatty liver (refer to Non-Patent Document 12). Pathological conditions similar to those in humans can be developed by artificially inducing a hypoxemic state in an animal with nonalcoholic fatty liver (refer to Non-Patent Document 13).

### Patent Document 1: Japanese Patent Laid-Open No. 2005-160415

### Patent Document 2: Japanese Patent Laid-Open No. 11-199477

### Patent Document 3: National Publication of International Patent Application No. 2005-510501

### Patent Document 4: Japanese Patent Laid-Open No. 2006-69911

### Patent Document 5: Japanese Patent Laid-Open No. 2006-151937

Non-Patent Document 1: Ludwig J. et al., Mayo Clin. Proc., 55, 434-438 (1980)

### Non-Patent Document 2: Matteoni C. A. et al., Gastroenterology, 116, 1413-1419 (1999)

### Non-Patent Document 3: Day C. P and James O. W., Gastroenterology, 114, 842-845 (1998)

### Non-Patent Document 4: Nishihara T. et al., Journal of the Japanese Society of Gastroenterology, 99, 570-576 (2002)

### Non-Patent Document 5: Reid A. E., Gastroenterology, 121, 710-723 (2001)

### Non-Patent Document 6: Weltman M. D. et al., Hepatology, 27, 128-133 (1998)

Non-Patent Document 7: Leclercq I. A. et al., J. Clin. Invest. 105, 1067-1075 (2000)
Non-Patent Document 8: Zhang B. H., Weltman M. et al., J. Gastroenterol. Hepatology, 14, 133-137 (1999)

### Non-Patent Document 9: Koppe S. W. P., Sahai A., et al., J. Hepatology, 41, 592-598 (2004)

### Non-Patent Document 10: Fan J. G. et al., World J. Gastroenterol, 11, 5053-5056 (2005)

Non-Patent Document 11: Matsuoka, M., and Tsukamoto, H.: Stimulation of hepatic lipocyte collagen production by Kupffer cell-derived transforming growth factor beta: implication for a pathogenetic role in alcoholic liver fibrogenesis. Hepatology. 11: 599-605 (1990)
Non-Patent Document 12: Maeda H., Nakajima T., Onishi K., and Hosomi Y.: Frequency of nonalcoholic abnormal liver function in male patients with obstructive sleep apnea syndrome and causes of its aggravation. Journal of the Hyogo Medical Association, 47(2), 115-120 (2004)
Non-Patent Document 13: Hatipoglu U. Rubinstein I.: Inflammation and obstructive sleep apnea syndrome pathogenesis: a working hypothesis. Respiration. 70(6): 665-671 (2003)

### DISCLOSURE OF THE INVENTION

### Problems to be Solved by the Invention

Thus, to observe efficacy of a test material over a long period using a pathological model with an experimental animal of NASH, one of lifestyle-related diseases, it is necessary that a required number of the above-mentioned pathological model mammals can be supplied rapidly without requiring labor at a predetermined timing using as simple a production method as possible. So far, however, there has been no useful pathological model with an experimental animal that reproduces neither the human NASH condition nor a method for producing the same.

The present invention was accomplished in view of the above-described situations, and an object thereof is to provide a novel pathological model with an experimental animal reproducing human nonalcoholic chronic hepatitis and/or liver fibrosis and/or cirrhosis resulting from fatty liver caused by lifestyle habits, a method for producing the same, and a method of utilizing a novel pathological model with an experimental animal.

### Means for Solving the Problems

To achieve the foregoing object, the inventors of the present invention conducted various researches and found that a blood oxygen partial pressure was maintained at a low level due to development of methemoglobinemia, and a NASH pathological model with an experimental animal was produced by maintaining the blood oxygen partial pressure at a low level, or that 1) a blood oxygen partial pressure was maintained at a low level by breeding in a low-oxygen environment, and 2) a NASH pathological model with an experimental animal was produced by maintaining the blood oxygen partial pressure at a low level in an animal with fatty liver. Thus, the present invention was accomplished.

The pathological model with an experimental animal of the present invention (excluding human) keeps biochemical characteristics and/or histopathological characteristics of nonalcoholic steatohepatitis and/or liver fibrosis and/or cirrhosis generated by inducing an in-vivo hypoxic state or inducing an in-vivo hypoxic state based on breeding in a low-oxygen environment.

Similarly, to achieve the foregoing object, the method for producing a pathological model with an experimental animal of the present invention produces a pathological model with an experimental animal (excluding human) which keeps biochemical characteristics and/or histopathological characteristics of nonalcoholic steatohepatitis and/or liver fibrosis and/or cirrhosis by inducing an in-vivo hypoxic state or inducing an in-vivo hypoxic state based on breeding in a low-oxygen environment.

The present invention provides a pathological model with an experimental animal of NASH, of which useful pathological model with an experimental animal did not exist before. This pathological model with an experimental animal exhibits each progression stage of hepatitis and/or liver fibrosis and/or cirrhosis and/or liver cancer that progresses from fatty liver without administering alcohol.

Sleep apnea syndrome with fatty liver, which increases a risk of developing NASH in human patients, can be made similar to pathological conditions developed in human patients by artificially inducing a hypoxemic state in an animal with nonalcoholic fatty liver. Depending on breeding conditions, an experimental animal model with pathological conditions in progression can be produced which stably exhibits and keeps changes in biochemical parameters of the NASH condition and histopathological characteristics that have components described in Non-Patent Documents 1 to 10. Since this pathological condition is a progressive disease, it may show symptoms such as ascites, hemorrhagic tendency, and induction of hepatic encephalopathy and portal hypertensions associated with decreased portal vein blood flow, that is, symptoms such as esophageal varices, gastrointestinal tract hemorrhage, splenomegaly, and hepatic encephalopathy, which result from progressive irreversible loss of hepatic functions, the basic pathological condition of cirrhosis, specifically, impairment in production of albumin and blood coagulation factors (including prothrombin).

The present invention, provides a method characterized by comprising the step of breeding while inducing an in-vivo hypoxic state in an animal due to a blood oxygen partial pressure decreased by producing methemoglobin by administering nitrite and/or hydroxylamine which does not exhibit direct hepatotoxicity without administering alcohol, a chronic hepatitis inducing agent, or a cirrhosis inducing agent, and a NASH pathological model with an experimental animal produced by this method. More specifically, the present invention provides a method characterized by comprising the step of breeding while adjusting the degree of the hypoxemic state by adjusting the dose, the number of doses, and the treatment period, and a pathological model with an experimental animal having a condition similar to the human NASH condition, which is useful for studies and elucidation of progression processes of the pathological condition as well as development and research of agents for prophylactic and therapeutic treatments of progression of the pathological condition, screening for a bioactive substance functioning for these prophylactic and therapeutic treatments, and a method for these prophylactic and therapeutic treatments by using the NASH pathological model with an experimental animal produced by this method.

Depending on breeding conditions, an experimental animal model with pathological conditions in progression can be produced which stably exhibits and keeps changes in biochemical parameters and histopathological characteristics of the NASH condition that have components described in Non-Patent Documents 1 to 3. Since the pathological condition is a progressive disease, it may show symptoms such as ascites, hemorrhagic tendency, and induction of hepatic encephalopathy and portal hypertensions associated with decreased portal vein blood flow, that is, symptoms such as esophageal varices, gastrointestinal tract hemorrhage, splenomegaly, and hepatic encephalopathy, which result from progressive irreversible loss of hepatic functions, the basic pathological condition of cirrhosis, specifically, impairment in production of albumin and blood coagulation factors (including prothrombin).

The present invention provides a method characterized by comprising the step of breeding while developing methemoglobinemia by breeding an experimental animal having fatty liver in a low-oxygen environment without administering alcohol, a chronic hepatitis inducing agent, or a cirrhosis inducing agent and finally inducing an in-vivo hypoxic state in an animal due to a decreased blood oxygen partial pressure, and a NASH pathological model with an experimental animal produced by this method. More specifically, the present invention provides a method characterized by comprising the step of maintaining a hypoxemic state by adjusting the oxygen concentration, specifically, an environment of the breathing oxygen concentration in breeding, and a pathological model with an experimental animal having a condition similar to the human NASH condition, which is useful for studies and elucidation of progression processes of the pathological condition as well as development and research of agents for prophylactic and therapeutic treatments of progression of the pathological condition, screening for a bioactive substance functioning for these prophylactic and therapeutic treatments, and a method for these prophylactic and therapeutic treatments by using the NASH pathological model with an experimental animal produced by this method.

The pathological model with an experimental animal produced without administering alcohol exhibits at least one of characteristics in each progression stage of steatohepatitis and/or liver fibrosis and/or cirrhosis and/or liver cancer. It is known that, as described above, the basic pathological conditions of cirrhosis are progressive irreversible loss of hepatic functions and portal hypertension associated with a decreased portal vein blood flow, and the pathological model of the present invention may exhibit at least one of these characteristics.

In the present invention, an experimental animal with fatty liver (experimental animal having fatty liver) may be used as a starting test material.

Since fatty liver exists as a base of the human NASH condition, an animal having fatty liver was used as a starting experimental animal. The experimental animal with fatty liver can be produced by, for example, orally giving a methionine-deficient high fat diet or a choline-deficient high fat diet for a certain period (refer to Cheng Y. F. et al., Transplant., 71, 1221-1225 [2001] and Dong H. et al., Gastroenterol., 11, 1339-1344 [2005]). In the present invention, the method for producing an experimental animal with fatty liver itself is not limited.

In general, experimental animals with fatty liver can be identified by checking deposition of triglyceride in the liver and the triglyceride content in the liver, checking histopathologically macrovesicular steatosis in hepatocytes, checking biochemically leakages of enzymes (AST, aspartate aminotransferase; ALT, alanine aminotransferase) from the hepatocytes into plasma, and the like.

These methods and these pathological model with experimental animals provide methods and pathological model with experimental animals, which are useful for studies and analytical research of the mechanism of aggravating lifestyle-related diseases associated with hypoxemia as well as development and research of agents for prophylactic and therapeutic treatments of progression of the pathological condition or advancement of the pathological condition to a severe condition, screening for a bioactive substance functioning for these prophylactic and therapeutic treatments, and a method for these prophylactic and therapeutic treatments.

In the present invention, to produce the pathological model with an experimental animal produce, the above-mentioned blood oxygen partial pressure is preferably lower than 108 hectopascal, but the blood oxygen partial pressure can be achieved by maintaining the oxygen concentration in the breeding environment of the experimental animal at 180 hectopascal or lower. It is natural that the lower limit of the blood oxygen partial pressure and the duration of maintaining the blood oxygen partial pressure are at least within ranges in which an experimental animal can survive, and it is needless to say that it is necessary to maintain the oxygen concentration in the breeding environment of the experimental animal at a level sufficient to achieve the blood oxygen partial pressure. Thus, the present invention relates to a method for inducing inflammation that progresses to fibrosis by finally inducing an in-vivo hypoxic state in an animal with fatty liver, and a pathological model with an experimental animal. More specifically, the present invention provides a method for inducing the NASH condition by finally inducing an in-vivo hypoxic state in an animal with fatty liver by arbitrarily adjusting the oxygen concentration in the breeding environment of an experimental animal and the breeding period, and a NASH pathological model with an experimental animal.

Furthermore, the above-mentioned blood oxygen partial pressure is preferably lower than 108 hectopascal (hPa). It is natural that the lower limit of the blood oxygen partial pressure is within the range in which an experimental animal can survive. Thus, the present invention relates to a method for forming an in-vivo hypoxic state in an animal with fatty liver and inducing inflammation that easily progresses to fibrosis and a NASH pathological model with an experimental animal. More specifically, the present invention relates to a method for inducing a NASH condition by inducing an in-vivo hypoxic state in an animal with fatty liver by arbitrarily adjusting the dose, the number of doses, and the treatment period, and a NASH pathological model with an experimental animal.

The pathological model with an experimental animal produced without administering alcohol exhibits at least one of characteristics at each progression stage of steatohepatitis and/or liver fibrosis and/or cirrhosis and/or liver cancer. It is known that, as described above, the basic pathological conditions of cirrhosis are progressive irreversible loss of hepatic functions and portal hypertension associated with a decreased portal vein blood flow, and, naturally, the pathological model of the present invention may exhibit at least one of these characteristics.

In the present invention, an experimental animal with fatty liver (experimental animal having fatty liver) is used as a starting test material.

Since fatty liver exists as a base of the human NASH condition, an animal having fatty liver was used as a starting experimental animal. The experimental animal with fatty liver can be produced by, for example, orally giving a methionine-deficient high fat diet or a choline-deficient high fat diet for a certain period (refer to Cheng Y. F. et al., Transplant., 71, 1221-1225 [2001] and Dong H. et al., Gastroenterol., 11, 1339-1344 [2005]). In the present invention, the method for producing an experimental animal with fatty liver itself is not limited.

In general, an experimental animal with fatty liver can be identified by checking deposition of triglyceride in the liver and the triglyceride content in the liver, checking histopathologically macrovesicular steatosis in hepatocytes, checking biochemically leakages of enzymes (AST, aspartate aminotransferase; ALT, alanine aminotransferase) from the hepatocytes into plasma, and the like.

In the present invention, to maintain the blood oxygen partial pressure at a level lower than 108 hPa, less than 70% of hemoglobin in a target animal (experimental animal with fatty liver) may be converted to methemoglobin. Since the experimental animal with methemoglobinemia having 70% or higher of methemoglobin generally dies, the range not exceeding this level is desired, but the proportion of methemoglobin itself is not limited in the present invention.

In normal erythrocytes, the methemoglobin concentration is generally maintained at 1% or lowers by a balance between production and reduction of methemoglobin. Therefore, when production of methemoglobin increases, or, to the contrary, the reduction thereof is impaired, the balance is disrupted, leading to methemoglobinemia. In methemoglobinemia with the proportion of methemoglobin in the total blood hemoglobin level being 10% or higher, the oxygen supply becomes insufficient, inducing cyanosis symptoms. Methemoglobin cannot bind to oxygen or carry oxygen to the whole body. Furthermore, methemoglobin changes a property that oxygenated hemoglobin is dissociated to oxygen and hemoglobin and makes it difficult for oxygen to release from oxygenated hemoglobin that has reached a tissue, leading to anoxia of the tissue due to impairment of oxygen transport.

In the present invention, to develop methemoglobinemia having lower than 70% of methemoglobin, nitrites or hydroxylamine, which serves as a hypoxemia-inducing agent, is readily available from reagent-related manufacturers. Furthermore, in general, a methemoglobinemic condition can be identified by measuring levels of methemoglobin and/or hemoglobin in a blood sample of the experimental animal.

Unlike lipid soluble carbon tetrachloride or the like, nitrites or hydroxylamine, a water-soluble substance, is not a substance metabolized by liver microsomal cytochrome P-450 enzymes to be converted to a highly reactive metabolite, and does not serve as a substrate of metabolization by cytochrome P-450 enzymes or produce a highly reactive metabolite by oxidation which exhibits cytotoxicity and damages cells. Since neither of the substances requires oxidative metabolism in hepatocytes for excretion, toxicity directly against the liver can be ignored, and they are excellent as materials that develop methemoglobinemia by administration thereof and induce anoxic tissues.

The total dose of nitrites and/or hydroxylamine is 10 mg/kg/day or higher, but the range in which methemoglobinemia having 70% or more of methemoglobin is not developed is preferred as the dose of nitrites and/or hydroxylamine. 30 to 70 mg/kg body weight/day is preferred. When administered, the drug substance of nitrites and/or hydroxylamine is arbitrarily diluted in physiological saline, and this solution can be administered (preferably, intraperitoneal administration). The treatment period is 3 to 16 weeks, preferably 4 to 12 weeks. In the present invention, the treatment period can be changed depending on the type of the experimental animal, the administration concentration, the amount, and the administration site according to the purpose.

Examples of nitrites used in the present invention include ammonium nitrite, potassium nitrate, sodium nitrite, barium nitrite, and cesium nitrite. Examples of nitrite esters that can be used include isobutyl nitrite, isopentyl nitrite, ethyl nitrite, butyl nitrite, propyl nitrite, pentyl nitrite, and methyl nitrite, but nitrite esters are not particularly limited so long as they are in a molecule form that can be administered as a nitrous acid.

To administer nitrites and/or hydroxylamine, in addition to the above-mentioned physiological saline, for example, they may be mixed, diluted, and stabilized with oils and fats, saccharides, proteins, or the like. Therefore, in the present invention, forms of nitrites and/or hydroxylamine such as emulsification product, powder, tablet, and capsule, dosage forms, and the like are not limited and can be arbitrarily selected.

Administration methods such as oral administration, intraperitoneal administration, and intrarectal administration are not limited in the present invention. The level of methemoglobin in blood collected from the cervical vein of an animal positively correlates to the dose of nitrites or hydroxylamine, and generally the oxygen partial pressure in arterial blood collected from a cannula placed in the cervical artery of the animal negatively correlates to these doses, but is not limited in the present invention.

The present invention induces a hypoxemic state in an animal with fatty liver to induce the NASH condition. More specifically, a progression state and severity can be adjusted by giving repeated loads of a hypoxic state to an animal with fatty liver by adjusting the dose, the number of doses, and the treatment period. A desired pathological model with an experimental animal can be produced by breeding an experimental animal while administering a hypoxemia-inducing agent under such a condition. Breeding by methods other than the above-described administration can be performed according to any known breeding method depending on the experimental animal species.

In the present invention, it may be understood that the oxygen concentration in the breeding environment of a target animal (experimental animal with fatty liver) is maintained at 180 hectopascal or lower to maintain the blood oxygen partial pressure at a level lower than 108 hPa.

The concentration of oxygen reaching the alveoli is also decreased due to a decreased oxygen partial pressure in inspiration. Oxygen supply by breathing becomes insufficient, and the oxygenated hemoglobin concentration decreases, inducing cyanosis symptoms. Anoxic tissues are caused by the decreased oxygenated hemoglobin reaching tissues.

In the present invention, breeding equipment for adjusting the breathing oxygen concentration in the breeding environment of experimental animals required for induction of hypoxemia to 180 hectopascal or lower is readily available related manufacturers. Furthermore, in general, the insufficient oxygen supply to tissues can be identified by measuring amounts of an oxygen partial pressure and hemoglobin in a blood sample of an experimental animal by a usual method.

In the present invention, a NASH pathological model with an experimental animal is produced by 1) breeding in a low-oxygen environment to maintain the blood oxygen partial pressure at a low level and 2) maintaining the blood oxygen partial pressure at a low level as described above, and NASH pathological model with an experimental animal having different properties can be produced by administering arbitrary substances that directly or indirectly function in the process of the production of the experimental animal, such as chronic hepatitis-inducing agents, cirrhosis-inducing agents, agents converting hemoglobin to methemoglobin, hypoxemia-inducing agents, and in-vivo oxidation-promoting agents solely or in combination to the experimental animal.

The present invention induces a hypoxemic state in an animal with fatty liver by breeding in a low-oxygen environment to induce the NASH condition. More specifically, the progression state and the severity of the pathological condition can be adjusted by giving repeated loads of a hypoxic state to an animal with fatty liver by adjusting the oxygen concentration and the breeding period. Furthermore, by breeding an experimental animal while administering an arbitrary substance such as a chronic hepatitis-inducing agent, a cirrhosis-inducing agent, an agent converting hemoglobin to methemoglobin, a hypoxemia-inducing agent, or an in-vivo oxidation promoting agent under such a condition, a desired pathological model with an experimental animal can be produced. Breeding by methods other than the above-described administration can be performed according to any known breeding method depending on the experimental animal species

As a method for judging whether the target experimental animal has characteristics of the NASH condition or not, for example, estimated diagnosis by blood biochemistry including plasma hyaluronic acid concentrations, AST and ALT activities, alkaline phosphatase (ALP) activity, bilirubin levels, cholinesterase activity, and albumin levels and definite diagnosis by liver biopsy, histopathological examination involving observation of macrovesicular steatosis in liver tissues obtained from the final sample, degeneration or necrosis of hepatocytes, periportal leukocytic infiltration into portal region, and the like can also be made.

Mammals to be used in the present invention are preferably mammal experimental animals for medical research that are commercially available from experimental animal suppliers or the like.

Examples of rodents to be used in the present invention include mice, rats, guinea pigs, and hamsters, and rats are particularly preferred. Wistar rat is more preferred. Examples of non-rodent mammals to be used in the present invention include rabbits, swine, and dogs, and a more preferred example is swine, which has the cardiovascular system and organs and tissues similar to those of humans, and miniature pigs and micro pigs are more preferred.

The pathological model with an experimental animal obtained in the present invention can be subjected to development of agents for prophylactic and therapeutic treatment of becoming severe of nonalcoholic steatohepatitis and/or liver fibrosis and/or cirrhosis. Specifically, it is natural that pathological model with an experimental animal obtained by the present invention can be utilized for development of substances effectively function as agents for prophylactic and therapeutic treatment thereof in the process of becoming severe from nonalcoholic fatty liver to hepatitis, liver fibrosis, and cirrhosis.

The pathological model with an experimental animal obtained in the present invention can be subjected to screening for a bioactive substance using hepatitis and/or liver fibrosis and/or cirrhosis as an indicator. That is, since simple and inexpensive animal experiment is enabled, efficient screening for a bioactive substance effective for the pathological condition is enabled.

For the above-described reasons, the pathological model with an experimental animal obtained in the present invention can be utilized in analysis of mechanisms advancing lifestyle-related diseases associated with hypoxemia as well as development of therapeutic agents and therapies.

Furthermore, the pathological model with an experimental animal of the present invention is characterized in that the above-mentioned pathological model with an experimental animal is subjected to development of agents for prophylactic and therapeutic treatment of progression of nonalcoholic steatohepatitis and/or cirrhosis, that the above-mentioned pathological model with an experimental animal is subjected to screening for a bioactive substance using nonalcoholic steatohepatitis and/or cirrhosis as an indicator, and that the above-mentioned pathological model with an experimental animal is used in analysis of mechanisms of developing lifestyle-related diseases associated with hypoxemia and development of agents for prophylactic and therapeutic treatment of progression of severity and development of therapies.

As explained above, according to the present invention, a model animal that substantially stably exhibits and keeps histopathological characteristics and biochemical characteristics of NASH can be obtained by performing a simple treatment in an animal with fatty liver prepared without administering alcohol using a production method comprising loading a hypoxic state similar to the mechanisms of developing and advancing the human NASH condition, and the required number of the above-mentioned NASH pathological model with experimental animals can be supplied rapidly without requiring labor at a predetermined timing.

By treating an animal with a dose, a number of doses, and a treatment period in treatment which are required for induction of hypoxemia, pathological model with experimental animals at various progression stages of the progressive NASH condition can be supplied, and/or a method for producing the same can be provided. Furthermore, pathological model with experimental animals at various progression stages of the NASH condition can be supplied by adjusting the oxygen concentration and the breeding period in treatment for inducing hypoxemia, that is, induction of an in-vivo hypoxic state based on breeding in a low-oxygen environment, and/or a method for producing the same can be provided. Medical elucidation of the progression mechanism of the pathological condition, which is a progressive disease from fatty liver to steatohepatitis, fibrosis, and cirrhosis, is enabled by the pathological model with an experimental animal of the present invention having mechanisms similar to those of developing and advancing the human NASH condition.

For development and establishment of new therapeutic agents for NASH and agents for preventing progression of severity and/or functional foods for decreasing a risk of developing NASH and functional foods for decreasing a risk of progression of severity and/or a method for treating NASH and a method for preventing progression of severity, it is very critical to check how sites of steatohepatitis, liver fibrosis and cirrhosis are affected, and it is required to observe effects of the above-mentioned test materials over a long period. The NASH pathological model with an experimental animal of the present invention enables development and research of agents for prophylactic and therapeutic treatment of progression of severity, screening for bioactive substance, materials, and methods effective and useful for prophylactic and therapeutic treatment of progression of severity, and higher quality and speed thereof is enabled.

### ADVANTAGES OF THE INVENTION

As explained above, according to the present invention, a pathological model with an experimental animal that keeps biochemical characteristics and/or histopathological characteristics of nonalcoholic steatohepatitis and/or cirrhosis was to be produced by inducing an in-vivo hypoxic state. As a result, a model animal can be obtained that substantially stably exhibits and keeps histopathological characteristics and biochemical characteristics of NASH, and the required number of the above-described NASH pathological model with experimental animals can be supplied rapidly without requiring labor at a predetermined timing.

Furthermore, according to the present invention, a pathological model with an experimental animal was to be produced in which an in-vivo hypoxic state was induced by breeding in a low-oxygen environment, and finally biochemical characteristics and/or histopathological characteristics of nonalcoholic steatohepatitis and/or cirrhosis were maintained. As a result, a model animal can be obtained that substantially stably exhibits and keeps histopathological characteristics and biochemical characteristics of NASH, and the required number of the above-described NASH pathological model with experimental animals can be supplied rapidly without requiring labor at a predetermined timing.

### BEST MODE FOR CARRYING OUT THE INVENTION

Hereafter, the present invention will be described more specifically with reference to the examples. However, the following examples should be construed as being an aid for obtaining concrete knowledge of the present invention but in no way limit the scope of the present invention.

### EXAMPLES

### <Preparation of Experimental Animal Having Nonalcoholic Fatty Liver>

An experimental animal having fatty liver was prepared according to the following procedure.

### 1) Experimental Animals

Experimental breeding was started using 6-week-old Wistar rats (Shimizu Laboratory Supplies, Co. Ltd.). The animals were bred in an environment with a 12-h (7:00-19:00) light-dark cycle, a humidity of 50 to 60%, and a temperature of 23° C. while given a feed and water ad libitum.

### 2) Materials and Methods

Preparation of animals with nonalcoholic fatty liver:

The rats were bred using a standard MF diet (Oriental Yeast Co., Ltd.) or a choline-deficient high-fat diets (8.000% vitamin-free casein, 37.950% lard, 48.375% sucrose, 4.000% Harper Mineral, 1.050% vitamin mix, 0.625% L-cystine w/w: Oriental Yeast Co., Ltd., hereinafter referred to as CDHF diets).

Deposition of triglyceride in liver:

After breeding animals using an MF diets or a CDHF diets for one month, the animals underwent laparotomy under ether anesthesia. The triglyceride content in the isolated liver (mg/g liver wet weight) was 12.1±1.1 mg/g in the MF fed group and 45.0±5.0 mg/g in the CDHF fed group, and it was found that deposition of triglyceride in the liver was significantly (p<0.01) induced.

Histopathological characteristics: The liver tissues collected from the rats after the above-described feeding and treatment were fixed overnight at in 10% formaldehyde in phosphate-buffered saline, then were dehydrated and embedded in paraffin. Sections (4 μm) of liver tissue were stained with hematoxylin and with Masson trichrome (MT) stain were observed under an optical microscope. Normal liver tissues having regular hepatocyte cords were noted in the liver of rats bred using the MF diets. Macrovesicular steatosis were observed in hepatocytes in most parts of the liver in rats bred using the CDHF diets for the same period. Based on the results of the examination of triglyceride accumulation in the previous section and histopathological examination of this section, it was confirmed that an animal with nonalcoholic fatty liver was produced.

Biochemical characteristics:

Biochemical markers reflecting hepatopathy, specifically, deviation of enzymes in the liver parenchyma cytoplasm (AST, aspartate aminotransferase; ALT, alanine aminotransferase) into blood: a plasma sample prepared by the centrifugal separation of a blood sample collected from the portal vein before the above-described isolation of the liver, AST activity increased significantly (p<0.05) to 66.2±8.1 KU/mL in the CDHF fed group compared to 53.2±6.2 KU/mL in the MF bred group and, and ALT activity showed no significant difference, with 13.9±1.6 KU/mL in the MF fed group and 17.6±2.3 KU/mL in the CDHF fed group.

Changes in plasma hyaluronic acid concentration:

The hyaluronic acid concentration in a plasma sample collected from the portal vein before the above-described isolation of the liver was measured to examine liver fibrosis. The hyaluronic acid concentration (ng/mL plasma) showed no significant difference, with 87.9±7.1 ng/mL in the MF fed group and 89.9±9.9 ng/mL in the CDHF fed group.

In the above biochemical examination and pathological examination, a time period required for production of an experimental animal having fatty liver was determined. When a CDHF diets was used, an animal with fatty liver was produced by breeding for 3 to 5 weeks.

### Example 1

Methemoglobin formation in blood by administration of sodium nitrite and decreases in blood oxygen partial pressure: After eight rats per group were bred using an MF diets or a CDHF diets for one month as preliminary breeding, the animals in each group were equally divided into two groups to form four groups each having four animals, and breeding was continued while giving the same feed as in the preliminary breeding period. Then, a low oxygen stress load test was started by methemoglobinemia induced by administering a sodium nitrite solution. Specifically, a solution of 50 mg/kg/day of sodium nitrite in physiological sodium or an equal volume of physiological saline was intraperitoneally administered to rats given an MF diets or a CDHF diets.

Using blood samples collected from a cannula placed beforehand in the carotid artery of rats, changes in levels of hemoglobin converted to methemoglobin (hereinafter referred to as methemoglobin) were checked over time, specifically, at 15 and 30 min and 1, 2, 3, 4, 5, and 6 h after administration of the sodium nitrite solution. The blood methemoglobin levels reached the local maximum values of 4.6 to 5.5 g/dL at 15 min after the intraperitoneal administration of 50 mg/kg of sodium nitrite solution (pH 7.4), 4.30 g/dL at 30 min, 2.93 g/dL at 1 h, 1.70 g/dL at 2 h, 1.0 g/dL at 3 h, 0.52 g/dL at 4 h, and 0.22 g/dL at 5 h, and returned to 0.16 g/dL, the baseline level before administration, within 6 h. After the intraperitoneal administration of physiological saline alone, the levels were maintained at the baseline level. The total hemoglobin levels were within the range of 15.4 to 16.6 g/dL.

The above-mentioned arterial blood oxygen partial pressure (hPa) in the blood sample inversely correlated to the methemoglobin levels. It reached the minimum values of 60 to 66 hPa at 15 min after administration of a sodium nitrite solution, 70.5 hPa at 30 min, 82.7 hPa at 1 h, 94.4 hPa at 2 h, 100.7 hPa at 3 h, 105.2 hPa at 4 h, 107.7 hPa at 5 h, and 108.3 hPa at 6 h, and returned to the normal range within 6 h. After administration of physiological saline alone, the arterial blood oxygen partial pressures were maintained at levels within the normal range.

Grouping animals according to feeding and treatment: Hereafter the groups of animals according to the above-mentioned feed used for breeding and treatment are designated as follows: Normal control group, MF diets plus intraperitoneal administration of physiological saline; CDHF group, CDHF diets plus intraperitoneal administration of physiological saline, CDHF plus nitrous group, CDHF diets plus intraperitoneal administration of a solution of sodium nitrite in physiological saline, control plus nitrous group, MF diets plus intraperitoneal administration of a solution of sodium nitrite in physiological saline.

Changes in histopathological and biochemical markers in animals produced by intraperitoneally administering a solution of 50 mg/kg/day of sodium nitrite in physiological saline or an equal volume of physiological saline to male Wistar rats having fatty liver or normal liver for one month will be explained. The assay results are expressed with the mean value among four animals in each group and standard error. Following one-way analysis of variance (ANOVA), Dunnett's test was used to compare the mean values between groups. Only statistical significant differences in comparison between the CDHF plus nitrous group and the CDHF group, of which animals were bred for the same period without receiving treatment to decrease the blood oxygen concentration, are shown.

Compared to that of normal control group, 13.2±1.4 mg/g, the liver triglyceride content (mg/g liver wet weight) as an index of fatty liver was almost same in the control plus nitrous group at the value of 13.2±0.9 mg/g, and significantly increased to 66.5±8.3 mg/g in the CDHF group, and 57.3±7.1 mg/g in the CDHF plus nitrous group, after one month of each treatment.

To examine histopathological changes in the liver, the livers were collected from animals following one-month intraperitoneal administration of either 50 mg/kg/day of sodium nitrite dissolved in physiological saline or an equal volume of physiological saline. The liver tissues were fixed overnight at in 4% formaldehyde in phosphate-buffered saline, then were dehydrated and embedded in paraffin. Sections (4 μm) of liver tissue were stained with hematoxylin and with Masson trichrome (MT) stain and were observed under an optical microscope. Macrovesicular steatosis in hepatocytes, degeneration and necrosis of hepatocytes, and periportal leukocytic infiltration into portal region were observed in the CDHF plus nitrous group. As a result, histopathological characteristics of steatohepatitis and/or liver fibrosis characteristic to the NASH condition were observed, with fibrosis in the lobule. Mild macrovesicular steatosis in hepatocytes, degeneration of hepatocytes, and periportal leukocytic infiltration into portal region were observed in the CDHF group, but no histopathological change was observed in the normal control group and the control plus nitrous group.

Groups of rats were separately established, and a solution of 50 mg/kg/day of sodium nitrite dissolved in physiological saline or an equal volume of physiological saline was intraperitoneally administered for two months. No marked change was observed in the rat liver wet weight in the normal control group, the DHF group, and the control plus nitrous group. However, significant (p<0.05) atrophy was induced in the CDHF plus nitrous group, with 68±16.3% based on the mean liver wet weight of the these three groups as 100%, and the condition progressed to cirrhosis, a terminal stage of chronic liver diseases. Laparotomy showed some individuals with retained ascites.

The plasma ammonia concentrations in rats obtained by intraperitoneally administering a solution of 50 mg/kg/day of sodium nitrite in physiological saline or an equal volume of physiological saline for two months were significantly (p<0.05) increased, with 43.2±14.2 μg/dL in the normal control group, 66.7±20.5 μg/dL in the CDHF group, 55.1±17.4 μg/dL in the control plus nitrous group, and 112.8±30.6 μg/dL in the CDHF plus nitrous group.

The plasma hyaluronic acid concentration (ng/mL plasma) as a biochemical marker of liver fibrosis was examined using a plasma sample collected after a solution of 50 mg/kg/day of sodium nitrite in physiological saline or an equal volume of physiological saline was intraperitoneally administered for one month. The plasma hyaluronic acid concentrations increased significantly (p<0.01), with 83.3±8.6 ng/mL in the normal control group, 89.8±4.5 ng/mL in the control plus nitrous group, 117.4±12.5 ng/mL in the CDHF group, and 240.3±38.9 ng/mL in the CDHF nitrous group.

Based on changes in biochemical markers reflecting hepatopathy, deviation of enzymes from the liver parenchyma cytoplasm into blood was examined using a serum sample collected after a solution of 50 mg/kg/day of sodium nitrite in physiological saline or an equal volume of physiological saline was intraperitoneally administered for one month. AST activity increased significantly (p<0.01), with 49.8±8.1 KU/mL in the normal control group, 47.4±3.8 KU/mL in the control plus nitrous group, 112.6±16.5 KU/mL in the CDHF group, and 237.2±63.7 KU/mL in the CDHF plus nitrous group. ALT ACTIVITY increased significantly (p<0.05), with 12.6±1.5 KU/mL in the control group, 12.7±1.9 KU/mL in the control plus nitrous group, 20.9±5.5 KU/mL in the CDHF group, and 35.5±6.8 KU/mL in the CDHF plus nitrous group.

Deviation of hepatobiliary enzymes (ALP: alkaline phosphatase, γ-GTP: γ-glutamyl transpeptidase) into blood was examined using a serum sample collected after a solution of 50 mg/kg/day of sodium nitrite in physiological saline or an equal volume of physiological saline was intraperitoneally administered for one month. ALP (nmol p-nitrophenol produce/min/mL plasma) significantly (P<0.01) increased, with 89.71±3.6 nmol in the normal control group, 91.7±3.0 nmol in the control plus nitrous group, 156.2±3.9 nmol in the CDHF group, and 192.1±4.3 nmol in the CDHF plus nitrous group. γ-GTP (IU/L plasma) significantly (p<0.01) increased, with 1.13±0.20 IU/L in the normal control group, 0.89±0.17 IU/L in the control plus nitrous group, 1.12±0.12 IU/L in the CDHF group, and 4.15±1.44 IU/L in the CDHF plus nitrous group.

The serum bilirubin concentration (mg/dL serum) was examined using a serum sample collected after a solution of 50 mg/kg/day of sodium nitrite in physiological saline or an equal volume of physiological saline was intraperitoneally administered for one month. The serum bilirubin was below the detection limit in the normal control group, the control plus nitrous group, and the CDHF group, but significantly (p<0.01) increased to above the detection limit in the CDHF plus nitrous group, with 12.4±3.5 mg/dL.

The blood cholinesterase activity (μmol substrate hydrolyzed/min/mL plasma) and the serum albumin concentration (mg/mL serum), which show a protein synthesizing ability in the liver, an indicator of the hepatic functional reserve in chronic liver diseases, were examined using a plasma sample collected after a solution of 50 mg/kg/day of sodium nitrite in physiological saline or an equal volume of physiological saline was intraperitoneally administered for two months. The cholinesterase activity significantly (p<0.01) decreased, with 2.59±0.24 μmol in the normal control group, 2.45±0.38 μmol in the control plus nitrous group, 2.14±0.29 μmol in the CDHF group, and 1.34±0.33 μmol in the CDHF plus nitrous group. The serum albumin concentrations were 54.0±3.5 mg/mL in the normal control group, 53.0±5.1 mg/mL in the control plus nitrous group, 40.3±6.0 mg/mL in the CDHF group, and 37.9±6.0 mg/mL in the CDHF plus nitrous group.

The content of nonheme iron, which is involved in fibrosis and is also closely associated with oxidative stress, was examined using serum and liver samples collected after a solution of 50 mg/kg/day of sodium nitrite in physiological saline or an equal volume of physiological saline was intraperitoneally administered for one month. The serum concentration significantly (p<0.01) increased, with 69.3±9.2 μg/dL in the normal control group, 72.1±7.5 μg/dL in the control plus nitrous group, 70.6±9.5 μg/dL in the CDHF group, and 118.7±8.2 μg/dL in the CDHF plus nitrous group. The nonheme iron content in the liver (μg/g liver wet weight) significantly (p<0.01) increased, with 120.0±9.1 μg/g in the normal control group, 126.7±6.1 μg/g in the control plus nitrous group, 158.1±19.3 μg/g in the CDHF group, and 293.7±18.7 μg/g in the CDHF plus nitrous group.

In the nutritional state in metabolic syndrome due to chronic excessive caloric intake, the basis of lifestyle-related diseases, it is estimated that active oxygen and free radicals are produced by energy metabolism in mitochondria, resulting in an increase in oxidative stress. It is assumed that, in the pathological model of the present application, in which fat accumulation and an insufficient oxygen supply are induced in cells in the liver, the principal organ of the nutrient metabolism, these mechanisms play important roles in progression to inflammation, liver fibrosis, and further to cirrhosis.

The amount of active oxygen and free radicals produced from energy metabolism in mitochondria was examined using mitochondria fractions isolated and prepared from the liver collected after a solution of 50 mg/kg/day of sodium nitrite in physiological saline or an equal volume of physiological saline was intraperitoneally administered for one month, as samples for electron spin resonance (hereinafter referred to as ESR) spectroscopic analysis. Specifically, 0.1% dodecyl maltoside, 5 mM glutamate, 5 mM malate, 100 mM succinate, mitochondria corresponding to 500 μg of proteins, 920 mM 5,5-dimethyl-1-pyrrolin-1-oxide (hereinafter referred to as DMPO), and 0.1 mM NADH-containing sample were incubated at 37° C. for 5 min, and ESR signals from an adduct of active oxygen and free radicals and DMPO were detected by ESR spectroscopic analysis. In the normal control group, the control plus nitrous group, and the CDHF group, active oxygen and free radicals were produced from mitochondria to the extent that only trace amounts of ESR signals from a spin adduct of DMPO and hydroxyl free radicals were detected. In the CDHF plus nitrous group, however, ESR signals from a spin adduct of DMPO and hydroxyl free radicals in the sample for measuring ESR from liver mitochondria were intensified to 3 to 5 times those in other groups, and production of active oxygen and free radicals from energy metabolism in mitochondria of the NASH model was found to be increased.

### Example 2

Experimental animals having fatty liver were prepared according to Example 1.

Production of blood methemoglobin and decreases in blood oxygen partial pressure after administration of hydroxylamine solution (hereinafter referred to as hydroxylamine solution) (pH 7.4):

After four animals per group were bred for one month using the above-mentioned MF diets or CDHF diets as preliminary breeding, breeding was continued using the same feed as in the preliminary breeding period, and then a low oxygen stress load was started by methemoglobinemia induced by administering a hydroxylamine solution. Specifically, a solution of 50 mg/kg/day of hydroxylamine in physiological saline adjusted to pH 7.4 or an equal volume of physiological saline was intraperitoneally administered to rats given the MF diets or the CDHF diets.

A cannula was placed beforehand in the carotid artery of rats, and production of methemoglobin converted from hemoglobin (hereinafter referred to as methemoglobin) was tracked using blood samples collected over time, at 15 and 30 min and 1, 2, 3, 4, 5, and 6 h, from the placed silicon tube. The methemoglobin levels reached the local maximum values of 3.8 to 4.4 g/dL at 15 min after intraperitoneal administration of 50 mg/kg/day of hydroxylamine solution, 3.50 g/dL at 30 min, 2.42 g/dL at 1 h, 1.38 g/dL at 2 h, 0.82 g/dL at 3 h, 0.42 g/dL at 4 h, and 0.20 g/dL at 5 h, and returned to 0.15 g/dL, the level before administration, the baseline level, within 6 h. After intraperitoneal administration of physiological saline alone, the methemoglobin levels were maintained at the baseline level. The total hemoglobin levels were within the range of 15.4 to 16.6 g/dL.

The arterial blood oxygen partial pressure (hPa) using this blood as a sample inversely correlated to the methemoglobin levels, and reached the local minimum values of 70 to 78 hPa at 15 min after administration of the hydroxylamine solution, 79.6 hPa at 30 min, 88.9 hPa at 1 h, 97.8 hPa at 2 h, 102.6 hPa at 3 h, 106.0 hPa at 4 h, 108.2 hPa at 5 h, and 109.3 hPa at 6 h, returning to the normal range within 5 h.

Designation of groups according to feed to animals and treatment:

Groups according to intraperitoneal administration treatment of 50 mg/kg/day of hydroxylamine solution to rats bred with the above-mentioned MF diets or CDHF diets are designated as the control plus hydroxylamine group and the NASH-hydroxylamine group. The normal control group and the CDHF group are as described in the relevant section in Example 1.

Changes in histopathological and biochemical markers in animals produced by intraperitoneally administering a solution of 50 mg/kg/day of sodium nitrite in physiological saline or an equal volume of physiological saline to male Wistar rats having fatty liver or normal liver for one month will be explained. The assay results are expressed with the mean plus or minus the standard error for four animals per each group. Following performing one-way analysis of variance (ANOVA), Dunnett's test was performed to compare the mean values between groups. Only statistically significant differences between the NASH-hydroxylamine group and the CDHF group, of which animals were bred for the same period without receiving treatment for decreasing the blood oxygen concentration, are shown.

The liver triglyceride content (mg/g liver wet weight) as an indicator of fatty liver was 13.2±1.4 mg/g in the normal control group, 13.6±1.3 mg/g in the control plus hydroxylamine group, 66.5±8.3 mg/g in the CDHF group, and 63.3±9.0 mg/g in the NASH-hydroxylamine group at one month.

To examine histopathological changes in the liver, the liver collected from animals treated with intraperitoneal administration of either 50 mg/kg/day of hydroxylamine solution or an equal volume of physiological saline for one month. Liver tissue was fixed in 4% formalin-phosphate buffer, then was dehydrated and embedded in paraffin. Sections (4 μm) of liver tissue were prepared by a usual method, then, were stained with the hematoxylin-eosin and Masson's trichrome stained and were observed under an optical microscope. Macrovesicular steatosis in hepatocytes, degeneration and necrosis of hepatocytes, and periportal leukocytic infiltration into portal region were noted in the NASH-hydroxylamine group. As a result, histopathological characteristics of steatohepatitis and/or liver fibrosis characteristic to the NASH condition were observed with fibrosis in the lobule. Liver fibrosis was slightly mild as compared with the CDHF plus nitrous group. Mild macrovesicular steatosis in hepatocytes, degeneration of hepatocytes, and periportal leukocytic infiltration into portal region were noted in the CDHF group, but no histopathological change was observed in the normal control group or in the control plus hydroxylamine group.

The changes in the biochemical markers tended to be similar to those in animals receiving administration of nitrites.

The amounts of reactive oxygen species and free radicals derived from mitochondrial energy metabolism tended to be similar to changes in animals receiving nitrites.

### Example 3

Breeding in a low-oxygen environment and decreases in blood oxygen partial pressure: After eight animals per group were bred with an MF diets or a CDHF diets in an environment in a cage with a normal air for one month as preliminary breeding, animals of each group were equally divided into two groups to form four groups each having four animals, and breeding was continued with the same diets as in the preliminary breed period. Then, animals were bred in a normal air or bred while being exposed to a hypoxic state by supplying a mixture gas into the breeding cage to establish a low-oxygen environment. Specifically, rats given the MF diets or the CDHF diets were divided into two groups, and one group each of the MF fed group and the CDHF fed group were bred in a cage with a normal air. Furthermore, another group each of the MF fed group and the CDHF fed group was bred in a cage into which nitrogen, oxygen, and carbon dioxide were supplied, with a composition of 79.01% or higher nitrogen, 20.95% or lower oxygen, and 0.04% or higher carbon dioxide.

The arterial blood oxygen partial pressure (hectopascal) in a blood sample collected from cannula placed beforehand in the carotid artery changed to 65 hectopascal in rats exposed to low oxygen for 1 h by supplying a mixture gas with a composition of 89.5% nitrogen, 10% oxygen, and 0.5% carbon dioxide and to 53 hectopascal in rats exposed to low oxygen for 1 h by supplying a mixture gas with a composition of 82.5% nitrogen, 15% oxygen, and 0.5% carbon dioxide, which decreased as compared with 102 hectopascal in control rats bred in a cage with a normal air. Thus, the arterial blood oxygen partial pressure decreased dependently on the level of the low oxygen given.

Male Wistar rats having fatty liver or normal liver were exposed to a hypoxic state under a condition of a gas with a composition of 94.5% nitrogen, 5% oxygen, and 0.5% carbon dioxide for 2 min, 10 times per 1 h, for 6 h everyday and bred for 1-2 months. Grouping according to feed to animals and low oxygen exposure: Hereinafter, groups according to the feed used for breeding the above-mentioned animals and the treatment are designated as follows: the normal control group, bred with an MF diets and an atmosphere environment; CDHF group, bred with a CDHF diets and an atmosphere environment; CDHF plus low oxygen group, bred with a CDHF diets and an environment with a low oxygen composition gas; control plus low oxygen group, bred with an MF diets and an environment with a low oxygen composition gas.

Changes in histopathological and biochemical markers in animals will be explained. The assay results are expressed with the mean plus or minus the standard error for four animals per each group. Following performing one-way analysis of variance (ANOVA), Dunnett's test was performed to compare the mean values between groups. Only statistically significant differences between the CDHF plus low oxygen group and the CDHF group, of which animals were bred for the same period without receiving treatment for decreasing the blood oxygen concentration, are shown.

After breeding with exposure to low oxygen or in a normal atmosphere for one month, the total hemoglobin level was 16.0±1.9 g/dL in the normal control group, 18.1±2.2 g/dL in the control plus low oxygen group, 15.8±2.1 g/dL in the CDHF group, and 17.7±2.3 g/dL in the CDHF low oxygen group.

The liver triglyceride content (mg/g liver wet weight) as an index of fatty liver was 13.2±1.4 mg/g in the normal control group, 16.6±2.9 mg/g in the control plus low oxygen group, 66.5±8.3 mg/g in the CDHF group, and 76.1±9.2 mg/g in the CDHF plus low oxygen group at one month.

Histopathological changes in the liver were examined. The isolated liver was fixed with 4% formalin-phosphate buffer, and paraffin sections were prepared according to a usual method. Then, the hematoxylin-eosin and Masson's trichrome stained sections were observed under an optical microscope, and rated with the following four grades depending on the severity of hepatopathy in the histopathological examination: A, no marked change ˜weak change; B, some pseudolobule formation (fibrosis); C, clear pseudolobule formation; and D, severe damage/a small number of surviving cells. A sample rated as C or D, which is similar to the image of human chronic hepatitis and/or cirrhosis, was determined as having histopathological characteristics of chronic hepatitis and/or cirrhosis. In the CDHF plus low oxygen group, macrovesicular steatosis in hepatocytes, degeneration and necrosis of hepatocytes, and periportal leukocytic infiltration into portal region were observed in the liver isolated at one month after the start of the low oxygen exposure, and as a result histopathological characteristics of steatohepatitis and/or liver fibrosis characteristic to the grade-C NASH condition was observed, with fibrosis in the lobule. The CDHF group was rated as A˜B, in which mild macrovesicular steatosis in hepatocytes, degeneration of hepatocytes, and periportal leukocytic infiltration into portal region were observed, but no histopathological change was observed in the normal control group or the control plus low oxygen group.

After breeding with exposure to low oxygen or in a normal atmosphere for two months, the plasma ammonia concentration (μg/dL) in rats tended to increase, with 43.2±14.2 μg/dL in the normal control group, 66.7±20.5 μg/dL in the CDHF group, 45.3±18.2 μg/dL in the control plus low oxygen group, and 84.1±25.7 μg/dL in the CDHF plus low oxygen group.

Using liver fibrosis as a biochemical marker, the blood hyaluronic acid concentration (ng/mL plasma) was examined in a plasma sample collected after breeding with exposure to low oxygen or in a normal atmosphere for one month. Compared to that of 83.3±8.6 ng/mL in the normal control group, the blood hyaluronic acid concentration did not differed in the control plus low oxygen group 79.9±6.1 ng/mL, and significantly (p<0.05) increased to 117.4±12.5 ng/mL in the CDHF group, and to 164.7±20.9 ng/mL in the CDHF plus low oxygen group.

Using changes in biochemical markers reflecting hepatopathy, deviation of enzymes in the liver parenchyma cytoplasm into blood was examined in a serum sample collected after breeding with exposure to low oxygen or in a normal atmosphere for one month. AST activity significantly (p<0.05) increased, with 49.8±8.1 KU/mL in the normal control group, 63.1±9.3 KU/mL in the control plus low oxygen group, 112.6±16.5 KU/mL in the CDHF group, and 176.7±37.1 KU/mL in the CDHF plus low oxygen group. ALT activity tended to increase, with 12.6±1.5 KU/mL in the control group, 14.3±1.7 KU/mL in the control plus low oxygen group, 20.9±5.5 KU/mL in the CDHF group, and 27.6±4.1 KU/mL in the CDHF plus low oxygen group.

Deviation of hepatobiliary enzymes (ALP: alkaline phosphatase, γ-GTP: γ-glutamyl transpeptidase) into blood was examined using a serum sample collected after breeding with exposure to low oxygen or in a normal atmosphere for one month. ALP (nmol p-nitrophenol produce/min/mL plasma) significantly (p<0.01) increased, with 89.71±3.6 nmol in the normal control group, 94.6±4.7 nmol in the control plus low oxygen group, 156.2±3.9 nmol in the CDHF group, and 211.1±14.3 nmol in the CDHF plus low oxygen group. γ-GTP (IU/L plasma) significantly (p<0.05) increased, with 1.13±0.20 IU/L in the normal control group, 1.29±0.22 IU/L in the control plus low oxygen group, 1.12±0.12 IU/L in the CDHF group, and 3.37±0.90 IU/L in the CDHF plus low oxygen group.

The serum bilirubin concentration was examined using a serum sample collected after breeding with exposure to low oxygen or in a normal atmosphere for one month. The serum bilirubin concentration was below the detection limit in the normal control group, the control plus low oxygen group, and the CDHF group but increased to above the detection limit with 7.7±24 mg/dL serum in the CDHF plus low oxygen group.

The serum albumin concentration (mg/mL serum), which shows protein synthesis ability in the liver as an indicator of the hepatic functional reserve in chronic liver diseases, was examined using a plasma sample collected after breeding with exposure to low oxygen or in a normal atmosphere for two months. The serum albumin concentration significantly (p<0.05) decreased, with 54.0±3.5 mg/mL in the normal control group, 50.0±4.3 mg/mL in the control plus low oxygen group, 40.3±6.0 mg/mL in the CDHF group, and 34.3±6.6 mg/mL in the CDHF plus low oxygen group.

The content of nonheme iron, which is involved in fibrosis and is also closely associated with oxidative stress, was examined using serum and liver samples collected after breeding with exposure to low oxygen or in a normal atmosphere for one month. The serum concentration (μg/dL) was 69.3±9.2 μg/dL in the normal control group, 66.2±5.7 μg/dL in the control plus low oxygen group, 70.6±9.5 μg/dL in the CDHF group, and 78.7±8.2 μg/dL in the CDHF plus low oxygen group. The nonheme iron content in the liver (μg/g liver wet weight) significantly (p<0.05) increased, with 120.0±9.1 μg/g in the normal control group, 117.9±7.1 μg/g in the control plus low oxygen group, 158.1±19.3 μg/g in the CDHF group, and 217±20.5 μg/g in the CDHF plus low oxygen group.

In the nutritional state in metabolic syndrome due to chronic excessive caloric intake, the basis of lifestyle-related diseases, it is anticipated that active oxygen and free radicals are produced by energy metabolism in mitochondria, resulting in an increase in oxidative stress. It is assumed that, in the pathological model of the present application, in which fat accumulation and an insufficient oxygen supply are induced in cells in the liver, the principal organ of the nutrient metabolism, these mechanisms play important roles in progression to inflammation, liver fibrosis, and further to cirrhosis.

The amount of active oxygen and free radicals produced from energy metabolism in mitochondria was examined using mitochondria fractions isolated and prepared from the liver collected after breeding with exposure to low oxygen or in a normal atmosphere for one month, as samples for electron spin resonance (hereinafter referred to as ESR) spectroscopic analysis. Specifically, 0.1% dodecyl maltoside, 5 mM glutamate, 5 mM malate, 100 mM succinate, mitochondria corresponding to 500 μg of proteins, 920 mM 5,5-dimethyl-1-pyrrolin-1-oxide (hereinafter referred to as DMPO), and 0.1 mM NADH-containing sample were incubated at 37° C. for 5 min, and ESR signals from an adduct of active oxygen and free radicals and DMPO were detected by ESR spectroscopic analysis. In the normal control group, the control plus low oxygen group, and the CDHF group, active oxygen and free radicals were produced from mitochondria to the extent that only trace amounts of ESR signals from the spin adduct of DMPO and hydroxyl free radicals were detected. In the CDHF plus low oxygen group, however, ESR signals from a spin adduct of DMPO and hydroxyl free radicals in the sample for measuring ESR from liver mitochondria were intensified to 2 to 3 times those in other groups, and production of active oxygen and free radicals from energy metabolism in mitochondria of the NASH model was found to be increased.

### Example 4

Experimental animals and the conditions of a breeding method according to Example 3 were used. Experimental animals having fatty liver were prepared by breeding with a high fat feed (37.950% lard, 48.375% sucrose, 4.000% Harper mineral, 1.050% vitamin mix, 0.625% L-cystine w/w: Oriental Yeast Co., Ltd., hereinafter referred to as high fat feed) and sucrose-added water for two months or longer.

Deposition of triglyceride in liver:

After breeding with a high fat feed for three months, the triglyceride content (mg/g liver wet weight) in the liver isolated by laparotomy under ether anesthesia was 12.1±1.1 mg/g in the MF-fed group and 26.0±5.2 mg/g in the CDHF breed group, and it was found that deposition of triglyceride in the liver was significantly (p<0.05) induced.

Histopathological changes in the liver with fibrosis, blood biochemical markers, and increased production of active oxygen and free radicals from energy metabolism in mitochondria were also induced by loading a low oxygen stress to an experimental animal having fatty liver prepared by a high fat diet according to Example 1, and the NASH model animal was produced.

1. A pathological model with an experimental animal (excluding human) keeping biochemical characteristics and/or histopathological characteristics of nonalcoholic steatohepatitis, which is produced by administering nitrites and/or hydroxylamine derivatives at a daily dose of 30 to 70 mg/kg body weight to an experimental animal having fatty liver for a treatment period of 3 to 16 weeks, more preferably 4 to 12 weeks to induce an in vivo hypoxic state with a blood oxygen partial pressure lower than 108 hectopascal through forming methemoglobin.

2. (canceled)

3. (canceled)

4. (canceled)

5. (canceled)

6. A pathological model according to claim 1, wherein the experimental animal is for medical research.

7. The pathological model according to claim 6, wherein the animal for medical research is a rodent.

8. The pathological model according to claim 7, wherein the rodent is a mouse or a rat.

9. (canceled)

10. A method for providing a pathological model with an experimental animal (excluding human), keeping-biochemical characteristics and/or histopathological characteristics of nonalcoholic steatohepatitis comprising the step of administering nitrites and/or hydroxylamine at a daily dose of 30 to 70 mg/kg body weight to an experimental animal having fatty liver for a treatment period of 3 to 16 weeks, more preferably 4 to 12 weeks to induce an in-vivo hypoxic state with a blood oxygen partial pressure lower than 108 hectopascal through forming methemoglobin.

11. (canceled)

12. (canceled)

13. (canceled)

14. (canceled)

15. The method according to claim 10, wherein the experimental animal is for medical research.

16. The method according to claim 15, wherein the animal for medical research is a rodent.

17. The method according to claim 16, wherein the rodent is a mouse or a rat.

18. (canceled)

19. (canceled)

20. A method for utilizing a pathological model with an experimental animal, according to claim 1, comprising one of the following steps: (1) subjecting the experimental animal to development of an agent for prophylactic treatment of progression of nonalcoholic steatohepatitis, (2) subjecting the experimental animal to development of a therapeutic agent for nonalcoholic steatohepatitis, (3) subjecting the experimental animal to screening for a bioactive substance using nonalcoholic steatohepatitis as an indicator, and (4) subjecting the experimental animal to analysis of the mechanism of developing a lifestyle-related disease associated with hypoxemia and development of an agent for prophylactic or therapeutic treatment of progression thereof and a therapy thereof.

21. (canceled)

22. (canceled)

23. (canceled)
